(19) United States
(12) Patent Application Publication
(10) Pub. No.: US 2008/0288071 A1
(43) Pub. Date: Nov. 20, 2008

(54) Expandable corpectomy device

(76) Inventors: Ashok Biyani, Sylavania, OH (US); Robert L. Doubler, Monroe, MI (US); John E. Hammill, SR., Maumee, OH (US)

Correspondence Address: MCHALE & SLAVIN, P.A., 2855 PGA BLVD, PALM BEACH GARDENS, FL, 33410, US

(21) Appl. No.: 11/748,176

(22) Filed: May 14, 2007

Related U.S. Application Data
(60) Provisional application No. 60/800,814, filed on May 16, 2006.

(51) Int. Cl. A61F 2/44

(52) U.S. Cl. 623/17.11

(57) ABSTRACT

The present invention describes an expandable vertebral implant and the method of use. The longitudinally expandable vertebral implant includes telescoping sections adapted for incremental expansion and ease of securement at any desired increment in situ, and constructed and arranged to engage opposing vertebrae, while simultaneously permitting easy insertion and removal of a similarly expandible top member.

### REFERENCE TO RELATED APPLICATIONS

This application claims benefit of the filing date of Provisional Application No. 60/800,814, filed on May 16, 2006, the contents of which are herein incorporated by reference.

### FIELD OF THE INVENTION

The invention generally relates to improvements in expandable vertebral implants and their methods of use; particularly, to a longitudinally expandable vertebral implant including telescoping sections configured for incremental expansion and ease of securement at any desired increment in situ, and constructed and arranged to engage opposing vertebrae, while simultaneously permitting easy insertion and removal of a similarly expandible top member.

### BACKGROUND OF THE INVENTION

The spine is formed by a flexibly arranged column of vertebrae divided into three sections (cervical, thoracic and lumbar). The vertebrae are separated by small cartilaginous cushions identified as intervertebral discs. Chronic back problems often manifest themselves due to a rupture or degeneration of these intervertebral discs either as a result of disease, injury or advanced age. When disc abnormalities occur, nerves within or adjacent to the spinal column may become inflamed or impinged resulting in the individual experiencing pain of varying degree and manifestation, diminished flexibility and reduced range of motion.

In order to reduce the pain associated with the movement of the intervertebral joint, surgical intervention is often indicated as a means to alleviate pressure upon the spinal cord while concomitantly stabilizing the associated vertebrae. This involves a surgical procedure to distract the disc and or vertebra, or portions thereof, and the insertion of bone fusing material into the cavity of the opposing vertebra. Corpectomy devices have been developed to help support the spine and maintain the normal spacing between opposing vertebrae. Some of these devices may be packed with fusing material to ensure solid bone growth between the two vertebrae.

Typically, corpectomy devices are pre-manufactured at various heights requiring that a cavity between opposing vertebrae be prepared and distracted to a dimension corresponding to the most suitably sized corpectomy device. The surgical procedure to prepare the implant site can be difficult and lengthy. Moreover, the procedure can increase risk of trauma to the tissues surrounding of the implant site.

Recently, distractable corpectomy devices have developed that may be used as both a fusion device and/or a means for maintaining intervertebral spacing. Often these implants include a drive means that allow the corpectomy device to be expanded in situ to a size that corresponds to the cavity created when the damaged tissue is removed. The drive means typically include devices such as gears, threaded rods, and the like, in mechanical engagement so as to expand or contract the device to a necessary distance between the vertebrae. Such constructions are complicated as they must necessarily comprise many moving parts, which likewise make them expensive to manufacture and more prone to failure. Moreover, when elongated to their expanded position, these devices often fail to provide a substantially enclosed cavity capable of retaining bone fusing material therein, which often used to ensure fusion between the two vertebrae.

### DESCRIPTION OF THE PRIOR ART

Although there are numerous patents directed to both artificial disk implants and expandable corpectomy devices, adapted for insertion and securement within the intervertebral space, the prior art nevertheless fails to teach an expandable corpectomy device which is capable of securely retaining bone growth/fusion materials therein, and which offers a means for simple incremental adjustment, such adjustment being conductible in situ, and thereby providing an individualized best fit, while providing a similarly adjustable and securable cover which, upon securement to the main body of the device, provides secure closure and retention of device engaging means effective for maintaining the device at a pre-selected degree of expansion. Moreover, the design of the instant invention is such that the contracting forces created by the surrounding vertebrae are distributed along the base of the device, not a gear mechanism, thereby reducing the probability of collapse of the device, in situ.

U.S. Pat. No. 5,171,278, to Pisharodi discloses an artificial disk implant and methods for implanting it, wherein the implant has a member for adapting, in size and shape, to an anatomical space between vertebrae and apparatus for expanding the member to conform to the space. Unlike the present invention, the design of this implant does not provide an internal cavity suitable for retention of fusing material to ensure solid bone growth between the two vertebrae.

U.S. Pat. No. 6,419,705 to Erickson is directed to expandable bone fusion devices and methods of use. In general, a fusion device according to the invention includes a first member and a second member which can be deployed and locked into an expanded configuration to stabilize the adjacent bone during fusion thereof.

U.S. Pat. No. 6,866,682 to An et al., describes a corpectomy device with an inner member telescopingly disposed in an outer member so that the inner member is movable in an axial direction. The inner and outer members are hollow, defining a chamber, and include apertures in communication with the chamber. A locking clip engages the inner and outer members to fix the position of the inner member with respect to the outer member. The longitudinal dimension of the device is adjustable by distracting the inner member so that the inner member extends from the outer member, and subsequently moving the locking clip from an unlocked position to a locked position. Again, unlike the present invention, the design of this implant includes multiple slots which, in the extended position, fail to provide an internal cavity capable of preventing fusing material therein from leaking, thereby resulting in impaired fusion at the insertion site. Moreover, the device requires the use of at least two separate locking means, and the device must be rotated and a set screw inserted therein to secure the locking clip at the desired longitudinal distance. This construction is problematic in that rotation of the implant in situ is difficult.

U.S. Pat. No. 7,029,498 to Boehm et al., disclose A height-variable vertebral body implant having a first, essentially U-shaped or C-shaped cage, and vertebral support surfaces formed on the first cage. The first cage is an inner cage, which is embraced and guided in a telescopic manner by a second, outer U-shaped or C-shaped cage. Further, legs of the inner and the outer cage are aligned such that a continuous lateral opening is obtained, and the inner and the outer cages are mutually fixed in a predetermined final position. The inner cage includes a longitudinally extending elongated hole having a unilateral toothing. In cooperation with an instrument including a complementary toothing, a relative movement and adjustment may be effected between the cages. In addition, a thread bore is formed in the outer cage in a position below the area of the elongated hole to fix a desired adjustment position. Again, this type of construction is undesirable given that it requires in situ alignment of a plurality of parts in constrained and difficult circumstances.

U.S. Patent Pub. No. 20060004447 to Mastrorio et al. is drawn to a height-adjustable device suitable for insertion between posterior spinal processes that allow the surgeon to post-operatively adjust the height of the implant. This reference is typical of devices which are fraught with mechanical deficiencies owing to their inclusion of a complicated drive means to separate the ends of the implant in communication with the vertebrae.

The aforementioned prior art disclose expandable vertebral implants constructed such that the majority of forces (contracting) from the tissue structures surrounding the joint are placed upon a single gear mechanism. This stress can eventually lead to cavitation of the device and possible damage to the vertebrae itself.

What has been heretofore lacking in the prior art is an expandable corpectomy device which offers:

1) a simple and easily operable means for effecting incremental expansion/contraction of the device;

2) permits such adjustment to be conducted in situ, thereby providing an individualized best fit;

3) provides a similarly adjustable and securable closure means for defining a cavity effective for the retention of bone growth/fusion material; and

4) upon securement to the main body, providing secure closure and retention of retention means, effective for maintaining the device at a pre-selected degree of expansion/contraction.

### SUMMARY OF THE INVENTION

The instant invention is related to a longitudinally adjustable corpectomy device and method for its use which is constructed and arranged to fit within the intervertebral distracted channel, and which provides a defined and secure cavity for bone growth/fusion material. Upon final assembly means for engaging the extendable members of the device, e.g. locking pins, are securely retained within the device by virtue of interaction with the securable closure means.

It an therefore an objective of the instant invention to provide a corpectomy device that may be adjusted either prior to insertion within the intervertebral cavity, or adjusted in situ within the cavity.

It is a further objective of the instant invention to provide an expandable corpectomy which includes means effective for enabling bone fusion after being implanted within the patient, such means illustratively represented as perforations, slits, or the like.

Yet another objective of the instant invention is to provide vertebra engageable endplates which may be constructed and arranged to accommodate various angular displacements of the cavity endpoints, and are effective to restore the normal curvature of the spine after the corpectomy device is installed.

Another objective of the present invention is to provide a device having a closable internal chamber for retention of bone growth/fusion means, such as fragments of bone, bone cement, or other material useful in facilitating the growth of bone.

Still a further objective of the invention is to teach a multi-function cover plate which is adjustable in length, securely engages the corpectomy device, and provides a means for securely retaining device locking means, illustratively disclosed as retaining pins, within the device.

These and other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is an upper perspective view of a corpectomy device, according to one embodiment of the instant invention, shown at its minimum length condition;

FIG. 2 is an upper perspective view of the corpectomy device of FIG. 1, shown at its maximum length condition;

FIG. 3 is an exploded view of the embodiment of FIGS. 1 and 2, illustrating the removable, expandable cover and locking means;

FIG. 4 is a top view of the corpectomy device shown without the cover;

FIG. 5 is a cross-sectional view of the corpectomy device as seen along the longitudinal axis of FIG. 4;

FIG. 6 is a partial, enhanced view of a portion of the slide mechanism;

FIG. 7 represents an illustrative embodiment of the end plate having a domed-shape with ridges for engaging the surface of the vertebrae;

FIG. 8A represents an alternative illustrative embodiment of the end plate having an angle faced end plate including spike-like protrusions for engaging the surface of the vertebrae;

FIG. 8B is an enhanced view of the spike-like protrusions shown in FIG. 8A;

FIG. 9 represents yet an additional illustrative embodiment of an endplate;

FIG. 10 is a side view of an angled endplate;

FIG. 11 is an upper perspective view of the sliding, expandable cover assembly with cover locking mechanism;

FIG. 12 is a cross-sectional view of the cover locking mechanism in the cover assembly.

FIG. 13 is a perspective view of the locking mechanism; and

FIG. 14 is an upper perspective view of various sizes of corpectomy devices.

FIG. 15 is a side view of the corpectomy device including physiology compensating members.

FIG. 16 is a perspective view of the corpectomy device shown in FIG. 15.

FIG. 17 is a perspective view of the corpectomy including physiology compensating members with the cover assembly removed.

FIG. 18 is a side view of the corpectomy device shown in FIG. 17.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

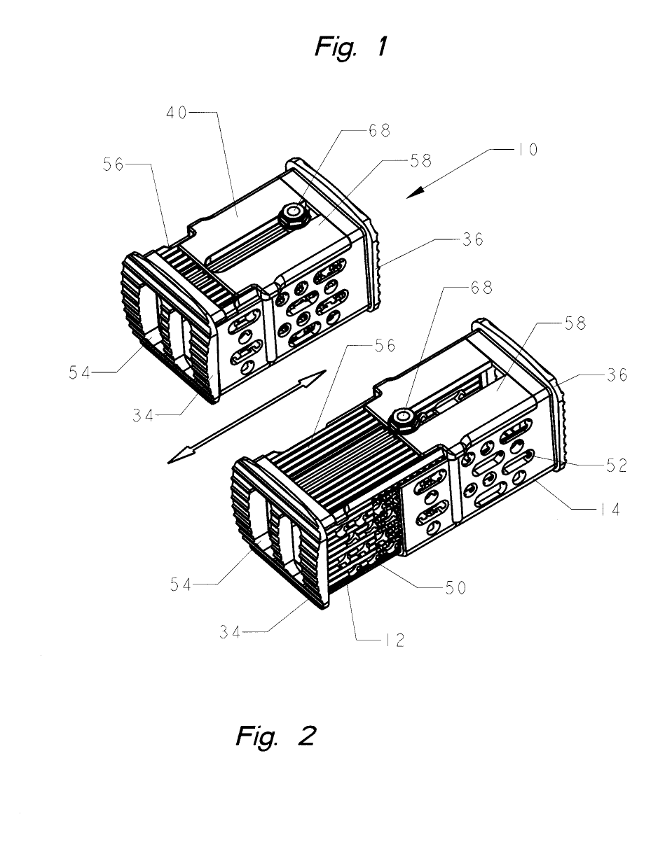

FIGS. 1 and 2 are upper perspective views of the fully assembled corpectomy device 10 (alternatively referred to as the implant device) in its minimum and maximum length condition, respectively. The instant invention could be used in all the spine levels (cervical, thoracic and lumbar).

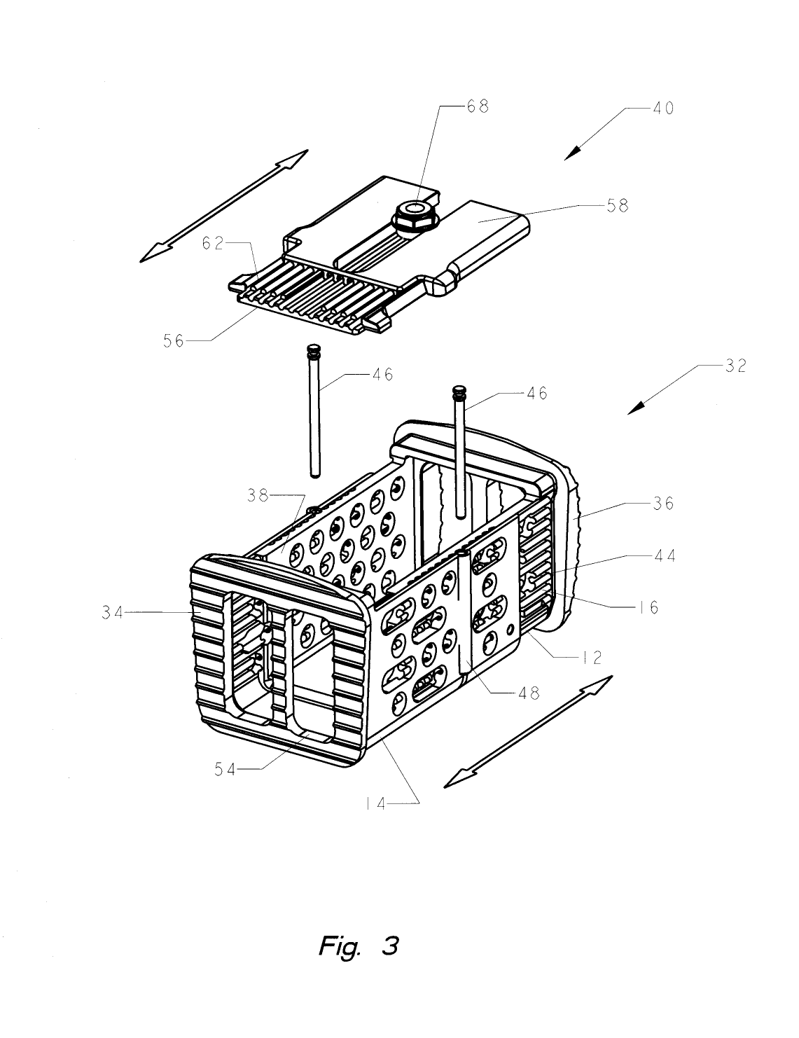

By way of an overview, the corpectomy implant device includes a base 32 formed by a first cavity defining member 12 telescopingly received in a second cavity defining member 14 and integrally connected first and second endplates 34 and 36 which define a hollow interior cavity 38 (see FIG. 3). The implant device includes an expandable and removable cover assembly 40 adapted to substantially enclose the interior cavity 38 to retain most of the bone fusion material (e.g., bone cement, bone chips, etc.) therein. The cover assembly includes a cover locking device that permits it to contract and expand relative to the overall longitudinal dimension of the device, as discussed further below.

The outer surface of the first member includes a plurality of integrally connected sidewall having a plurality of integrally formed longitudinal rail members 16 that interdigitate and slide along a plurality of longitudinal groove members 18 integrally formed on the outer surface of at least one sidewall of the second member, as shown in FIG. 5 and the partial, expanded view in FIG. 6. The constructions of the first and second members distribute the contracting forces created by the surrounding vertebrae along the base of the device, making it less prone to collapse. The first and second members are slidingly received such that the device can be telescoped incrementally to the desired length corresponding to the distance between adjacent lumbar vertebrae. Although not particularly shown, it is nevertheless herein contemplated that the outer surface of the sidewalls of the first member could include the plurality of groove members and the outer surface of the sidewalls of the second member could include the corresponding rail members without departing from the scope of the invention.

Even though the cross-section of the first and second members are shown and described herein as at least three integrally connected sidewalls (20, 22, 24) and (26, 28, 30) forming a square or rectangular cross-sectional base configuration (FIG. 5), it is hereby contemplated the device could be cylindrical, polygonal or the like, so long as the inner and outer member are able to telescope to the desired length.

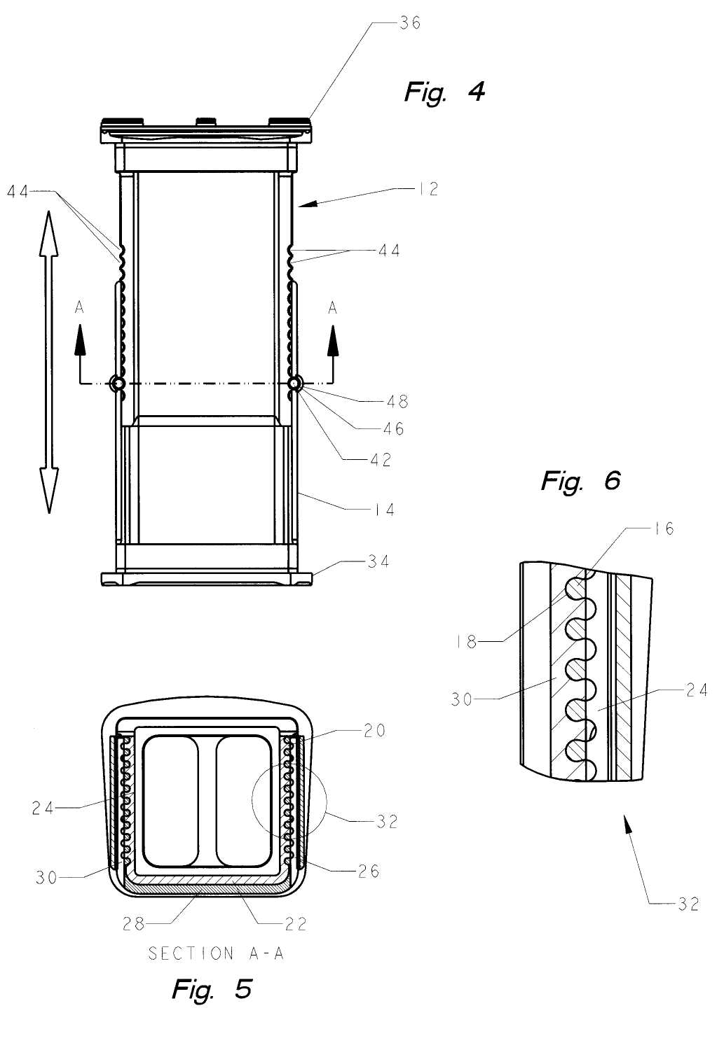

As best shown in FIGS. 4 and 5, the outer surface of opposing sidewalls (20, 24) of the first member include a plurality of integrally placed bores 42 formed transverse to the rail members. These bores are constructed and arranged to substantially conform to a portion of retaining pins 46 (see FIGS. 3 and 13). The inner surface of opposing sidewalls (26, 30) of the second member of the implant includes at least one integrally formed notch 48 constructed and arranged to receive the other portion of the pin not in contact with the bores 44. Thus when the notch 48 and one of bores 42 are aligned, the pin 46 is inserted therebetween and acts to removably and securably lock the first member relative to the second member. The pin includes an integrally connected head portion 56 (see FIG. 13) which allows the pin to be easily removed from the assembled device and adjusted to the desired longitudinal length. In a preferred embodiment, the plurality of bores are formed at approximately 1 mm increments along the longitudinal axis, so that the base may be telescoped to the necessary length between adjacent lumbar vertebrae.

As shown in FIGS. 1-3, the opposing sidewalls of the first and second member may include a plurality of means effective for enabling bone fusion (e.g., apertures, perforations, slits, or the like) 50, 52, formed therethough and constructed and arranged to facilitate the growth of bone, blood vessels and other tissue into the interior cavity of the device for enhanced bone fusion and stability, while also preventing the fusion material contained therein from seeping out into the body.

As shown in FIG. 5, the first and second member may include at least one solid sidewall 22, 28 for enhanced rigidity and strength of the implant against the contraction forces of neighboring tissues. Alternatively, it is contemplated herein that apertures similar to those formed in the opposing sidewalls of the first and second member could be formed.

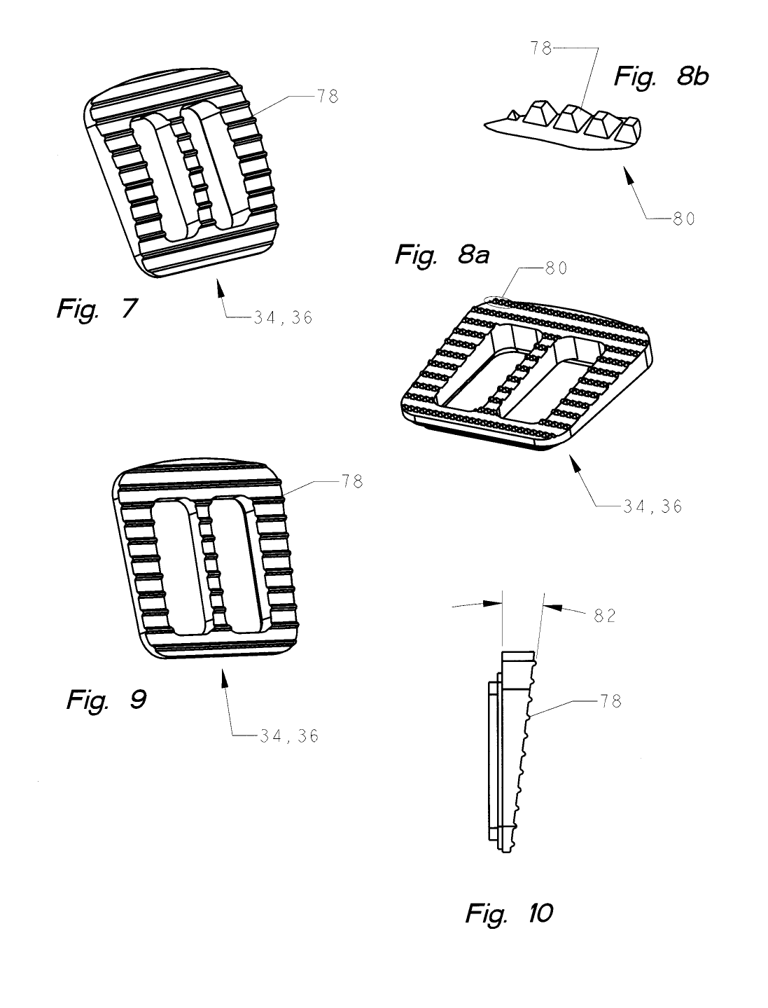

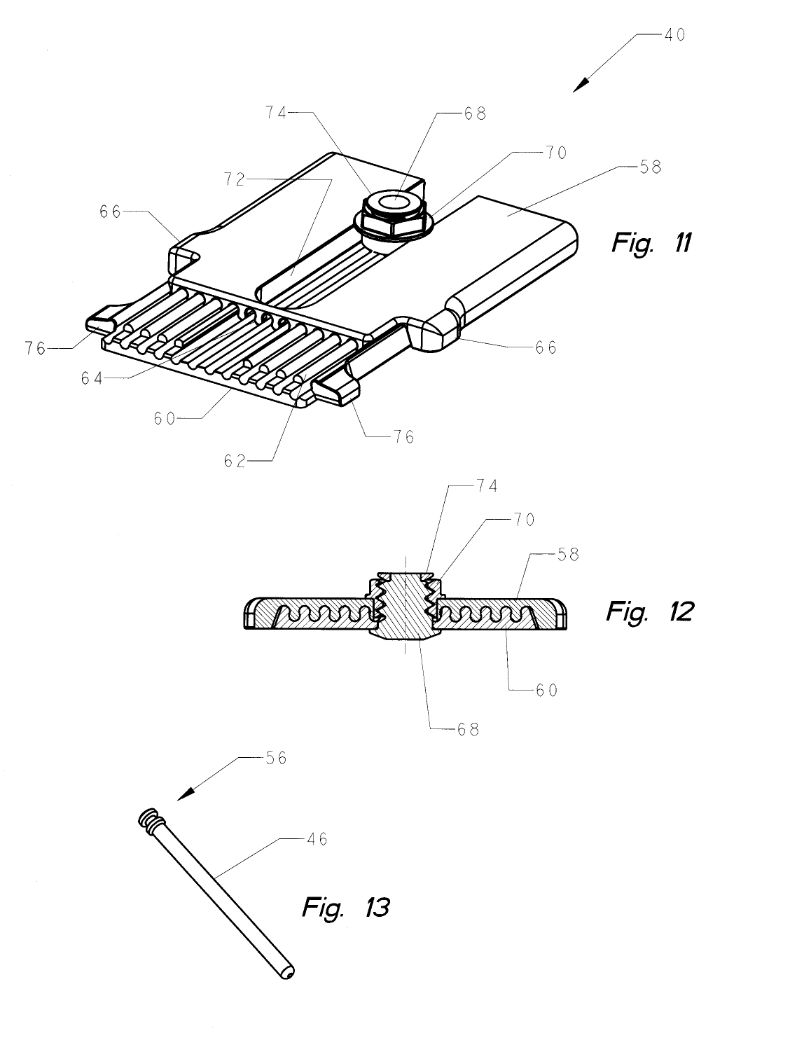

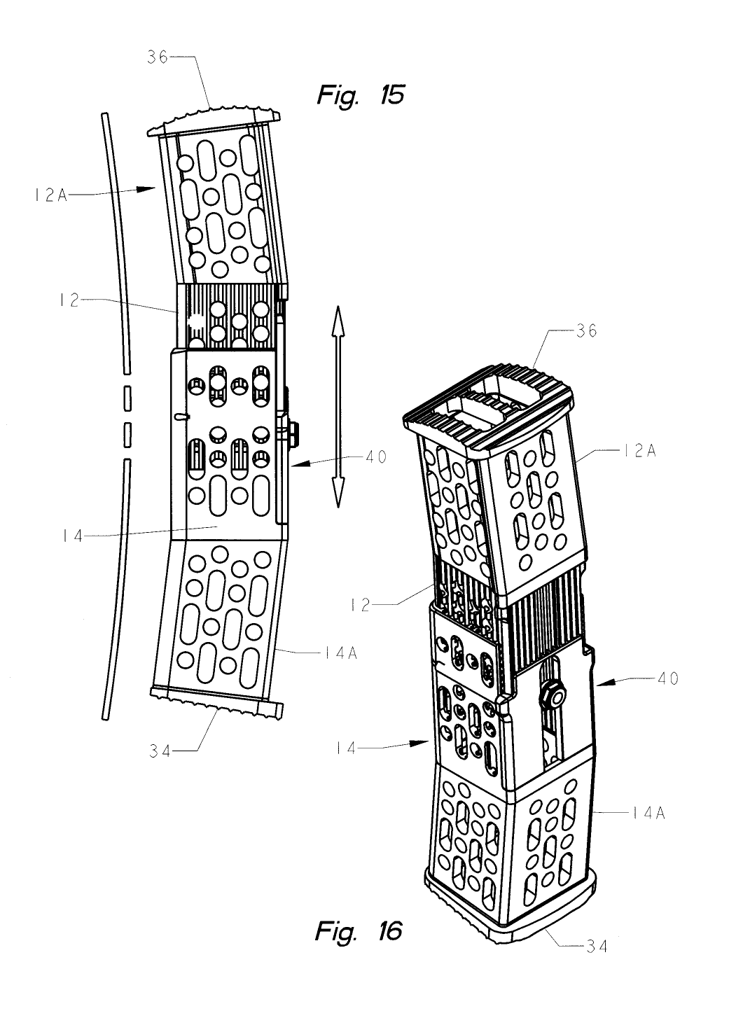

As shown in FIGS. 1-3, the base of the implant device includes opposing endplates 34, 36. The endplates may be interchangeably connected or permanently attached (laser welding) to the base of the corpectomy device. These endplates may be of any desired shape, size or thickness. For example, the endplate of FIG. 7 is dome-shaped and the endplate of FIG. 9 is substantially flat. In one embodiment shown in FIGS. 8A and 10, the endplates are formed at an angle 82 (e.g., 0, 3, 5 or 7 degrees) that will allow the implant to restore the normal curvature of the spine after the corpectomy device is installed. Moreover, the shape may or may not correspond to the cross-sectional shape and size (foot-print) of the base. In those instances where the patient presents unusual physiology, such as curvature of the spine (lordosis or kyphosis), additional physiology compensating members may be interposed between the first and second members and their respective endplates. These compensating members allow the corpectomy device to take on a more arcuate shape thereby conforming more closely with the existing spinal configuration as will be discussed in more detail with respect to FIGS. 15 through 18.

In a preferred embodiment the endplates have at least one aperture 54 formed therethrough for facilitating bone fusion with neighboring vertebrae. The surfaces in contact with the vertebrae may also include bone engaging means (e.g., teeth, ridges or keels, as shown in FIG. 8B). The engaging means should be constructed and arranged to help secure the implant to the vertebrae.

Referring now to FIGS. 11 and 12, illustrative, albeit non-limiting embodiments, are depicted of an adjustable cover assembly 40 used to substantially cover the internal cavity 38 of the base 32. The cover assembly includes an upper member 58 and a lower member 60, referred to as crossplates. In similar fashion to the first and second members of the base, the upper and lower crossplate members 58, 60 are constructed and arranged to telescope relative to each other, such that the cover can telescoped to the length of the base. That is, the inner surface of the upper member includes a plurality of integrally formed longitudinal cover rail members 62 that interdigitate and slide along a plurality of longitudinal cover groove members 64 integrally formed on the outer surface of the lower member, as shown in FIG. 11 and the cross-sectional view in FIG. 12. Conversely, the lower cover member could include the plurality of groove members and the upper cover member could include the corresponding rail members without departing from the scope of the invention.

As best illustrated in FIG. 12, the lower member of the cover includes an integrally attached threaded post 68 designed to remain stationary thereon. The upper member of the cover includes a slot 72 constructed and arrange to slidably receive the post when upper cover member is interlocked with the lower cover member. Once the overlapping cover members enclose the internal cavity of the base, securing means (shown here as an adjustable securing means, illustrated as a threaded nut 70 and a fixed securing means, illustrated as a locking washer 74) may be used to lock it in place along the slot. Moreover, the fixed securing means, e.g. the lock washer 74 may be fixedly positioned, e.g. adhered as by laser welding, to the post to further ensure that the cover remains stationary, and the nut 70 can not be inadvertently removed.

As shown in FIG. 11, the upper member 58 of the cover includes integrally formed and laterally projecting bosses 66 constructed and arranged on opposite sides to cover the head 56 of retaining pin 46, as illustrated in FIG. 13, when installed within the base of the device (see FIGS. 1 and 2). These bosses prevent the retention means (illustrated, albeit not limited to pins) from sliding out once the device is installed within the patient. The lower member is constructed and arranged to removably attach to the endplates or the base. As shown here, the attachment means are integrally formed and laterally projecting means for retaining the locking means, shown here as tabs 76 that are constructed and arranged to slide into corresponding slots in the endplates or base. Other means of attachment could be used without departing from the scope of the invention.

To adjust the instant corpectomy device to size in situ, the assembled base 32 would be placed into the cavity formed by the distraction of the disc and/or vertebra by a surgeon. Each endplate wall of the corpectomy device is placed in contact with the surfaces of the opposing vertebrae. The base should be positioned such that the internal cavity remains accessible to the surgeon. The base device is distracted to any desired increment by a distraction device. The first and second members of the base are then locked into position by insertion of the required retaining pin(s) into the base. In a preferred, albeit non-limiting embodiment two retaining pins are used. Then the surgeon may place the bone fusing material into the internal cavity by any suitable means. Lastly, the cover assembly is installed onto the base and slidably extended to enclose the internal cavity and the locking means tightened to lock the unit in place.

Alternatively, the instant corpectomy device may be expanded to the desired longitudinal length and filled with bone fusing material prior to being inserted into the cavity between the opposing vertebrae.

Suitable materials for any of the implant members may include any biologically compatible material, including, albeit not limited to, metal (e.g., titanium, steel), plastic, carbon or combinations thereof.

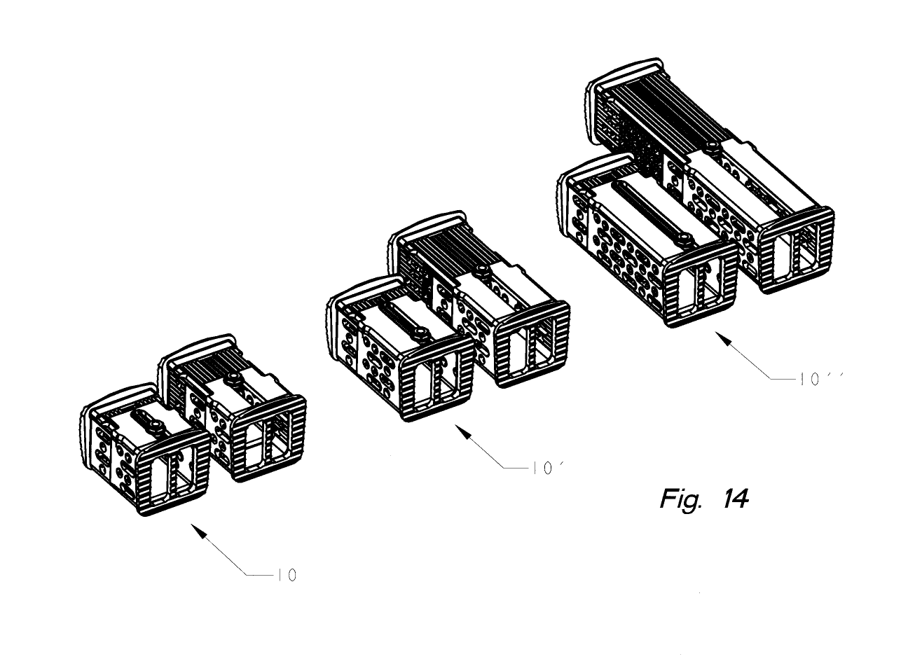

FIG. 14 is an upper perspective view of various sizes of corpectomy devices 10, 10′, 10″.

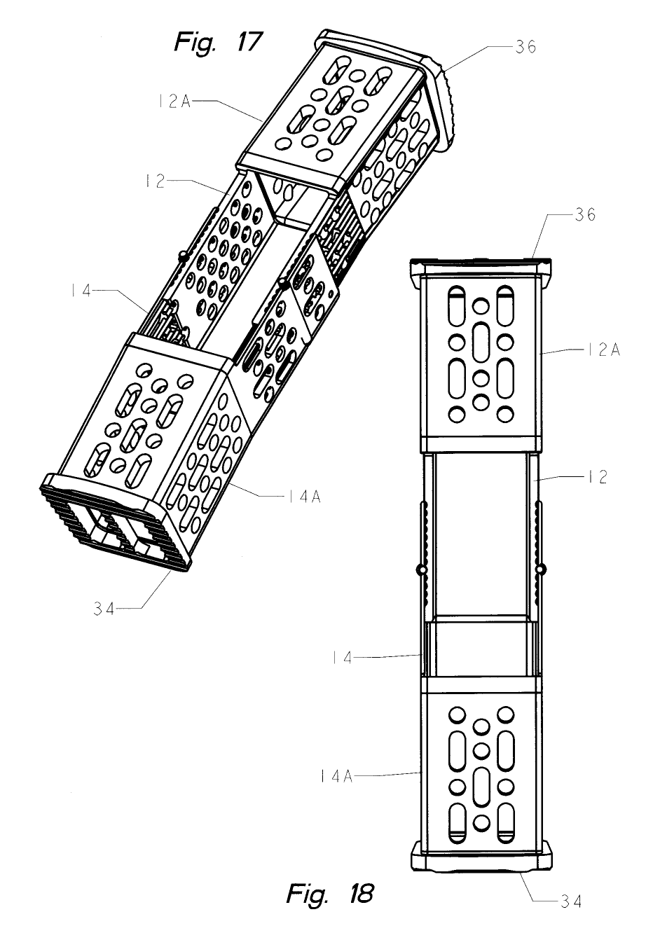

FIGS. 15 through 18 illustrate a corpectomy device particularly designed to be used in those patients suffering from abnormal curvature of the spine, also known as lordosis or hyperkyphosis. Lordosis is the abnormal concavity in the curvature of the lumbar and cervical spine as viewed from the side. kyphosis is the name given to the normal curvature of the human thoracic spine (looking at the spin from front to back, this curvature is concave). If for some reason, however, the curve becomes abnormally pronounced, the condition is referred to as hyperkyphosis—commonly known as Dowager's hump, or simply kyphosis. FIGS. 15 and 16 illustrate a corpectomy device with the cover assembly 40, a first member 12 a second member 14 and a first compensating member 12a and a second compensating member 14a as well as endplates 34 and 36. FIGS. 17 and 18 illustrate the same corpectomy device with the cover assembly removed. The compensating members 12a and 14a are joined to their respective first or second members (12 and 14) at a small angle, thereby resulting in a corpectomy device which is overall generally arcuate in shape. This arcuate shape provides much better results for those patients suffering from curvature of the spine.

Although the invention is described with reference to stabilization and fusion of adjacent spinal vertebrae, it is hereby contemplated that devices and methods disclosed herein could be used in all types of joints (ankle, interdigital, etc) found in the human or animal body.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A corpectomy implant device for retaining bone fusion material comprising:
a first cavity defining member having first opposing side walls spaced apart and integrally connected by a first bottom wall, and having a first end plate secured along a bottom edge of each said first side walls and said first bottom wall;
a second cavity defining member having second opposing side walls spaced apart and integrally connected by a second bottom wall, and having a second end plate secured along the bottom edge of each said second side walls and said second bottom wall;
said first and second cavity defining members being constructed and arranged for telescopic engagement to define an open-topped cavity of adjustable length;
at least one fixation means for securing a first member side wall to a juxtaposed second member side wall; and
a lid constructed and arranged to overlie said cavity and said at least one fixation means, said lid having a first crossplate having an end edge for engaging said first end plate and a second crossplate having an end edge for engaging said second end plate, said first and second crossplate constructed and arranged for telescopic engagement to form an adjustable length substantially equivalent to the length of said open-topped cavity;
wherein said first crossplate is secured to said second crossplate to close said open-topped cavity;
whereby bone fusion material is retained within said cavity.

2. The corpectomy implant device of claim 1, wherein said lid further includes a securing device effective for fixedly engaging said telescopingly engaged crossplates at a length substantially equivalent to the length of said open-topped cavity.

3. The corpectomy implant device of claim 2, wherein said securing device includes a slot in the upper crossplate, a post in the lower crossplate and a fastener engaged with said post to secure the upper and lower crossplates in a fixed position with respect to one another.

4. The corpectomy implant device of claim 1, wherein at least a portion of an opposing outer side wall of said first cavity defining member operatively engages at least a portion of an inner side wall of said second cavity defining member.

5. The corpectomy implant device of claim 4, wherein said operative engagement is via a plurality of interdigitating rails and grooves which extend longitudinally along at least a portion of an opposing outer side wall of said first cavity defining member and at least a portion of an inner side wall of said second cavity defining member.

6. The corpectomy implant device of claim 5, further including:
a plurality of substantially parallel bores spaced longitudinally along at least a portion of an opposing outer side wall of said first cavity defining member and at least a portion of an inner side wall of said second cavity defining member, each said bore extending transversely to said plurality of rails and grooves, and further including at least one notch formed on an inner side wall of said second cavity defining member extending in a direction transverse to said rails and grooves, and a retaining member which cooperates with at least one of said bores and said at least one notch to retain the first and second cavity defining members in a fixed telescopic relationship.

7. The corpectomy implant device of claim 6, wherein said lid includes a retention boss constructed and arranged to overlie an upper end of said retaining member to prevent removal thereof.

8. The corpectomy implant device of claim 1, wherein each said first and second end plates are interchangeably or permanently attached to each said first and second cavity defining members respectively, and each said endplate is selected to have a shape effective to restore a normal curvature to a spinal column

9. The corpectomy implant device of claim 1, wherein at least one of said first and second endplates has at least one aperture for facilitating bone fusion with neighboring vertebrae.

10. The corpectomy implant device of claim 6, wherein said retaining member is a pin which includes a head portion, wherein the pin is easily removed from a position where the pin engages one of said plurality of bores and said notch to permit said first and second cavity defining members to be telescoped relative to one another, subsequent to which said pin is reinserted into one of said bores and said notch to fix the telescoping relationship of said first and second cavity defining member.

11. The corpectomy implant device of claim 1 further including
at least one physiology compensating member interposed between the respective opposing sidewalls and bottom wall of a cavity defining member and its respective endplate.
